(19) United States
(12) Patent Application Publication
(10) Pub. No.: US 2007/0275088 A1
(43) Pub. Date: Nov. 29, 2007

(54) Conversion Of Sea-Shells And Other Calcite-Based And Aragonite-Based Materials With Dense Structures Into Synthetic Materials For Implants And Other Structures And Devices

(76) Inventor: Kenneth S. Vecchio, San Diego, CA (US)

Correspondence Address: FISH & RICHARDSON, PC, P.O. BOX 1022, MINNEAPOLIS, MN, 55440-1022, US

(21) Appl. No.: 11/838,181

(22) Filed: Aug. 13, 2007

Related U.S. Application Data
(63) Continuation of application No. PCT/US06/05175, filed on Feb. 13, 2006.
(60) Provisional application No. 60/652,228, filed on Feb. 11, 2005.

(51) Int. Cl. A61K 35/34; A61F 2/02

(52) U.S. Cl. 424/548; 423/308; 514/21; 623/23.72

(57) ABSTRACT

Bulk materials for implants and scaffolds made from hydrothermal conversion of bulk calcium carbonate materials with desired initial structures in order to utilize the mechanical and structural properties of the initial structures. Dense sea-shells, light-weighted sea urchin spines and strong marine bones such as cuttlebones are examples of bulk calcium carbonate materials with desired initial structures for producing various implants and scaffolds.

This application is a continuing application of co-pending PCT Application No. PCT/US06/05175 entitled “CONVERSION OF SEA-SHELLS AND OTHER CALCITE-BASED AND ARAGONITE-BASED MATERIALS WITH DENSE STRUCTURES INTO SYNTHETIC MATERIALS FOR IMPLANTS AND OTHER STRUCTURES AND DEVICES” and filed on Feb. 13, 2006 (PCT Publication No. WO 2006/086783) which claims the priority from U.S. provisional application No. 60/652,228 entitled “Conversion of Sea Shells and Other Calcite-Based Materials into Synthetic Bone Materials” and filed on Feb. 11, 2005.

The entire disclosures of the above patent applications are incorporated by reference as part of the specification of this application.

### BACKGROUND

This application relates to synthetic materials used for bone implants and other structures and devices.

A bone body part in a person or animal may deteriorate or become damaged due to illness, injury, aging and other factors. A deteriorated or damaged bone part may need repair or replacement. To fill bone defects or augment deficient bone, surgeons may use the patient's own bone, called an autograft, in a procedure that harvests the bone from a donor site, such as the pelvis. This autograft procedure is widely performed by surgeons, an autograft bone has significant limitations, including donor site complications, an inadequate amount or inappropriate shape of a harvested bone, and extremely high costs. Alternatively, surgeons can utilize allografts from a cadaver bone from a deceased person. However, allografts may cause undesired immune responses and transmit disease.

In addition to autografts and allografts, a synthetic bone material may be used as a bone replacement. Properly designed synthetic bone products may offer surgeons a broad range of bone grafting alternatives that mimic the architecture of human bones in a safe, sterile implant. A suitable material for bone replacements should have desired biocompatibility and material property compatibility with similar mechanical properties of natural bones. Replacement bone materials, when properly designed, should have various desired properties, such as an expected lifetime greater than that of the recipient, ability to survive in a body fluid environment, and ability to form a bone-implant interface with the body's natural tissues.

Many materials have been tested as bone replacements. For example, steel, titanium, and polymers have been used as replacement bone material in surgery. However, these and other materials do not have desired material properties to match the material properties of the natural bones.

As an example, the cortical bone forms the dense, load-bearing outer shell of human bones. The cortical bone have been measured to have a stiffness measured by Young's modulus in an approximate range from 7 GPa to 30 GPa, and a tensile strength in an approximate range from 50 MPa to 150 MPa, and one to three percent elongation at fracture. A bone replacement should be designed to match these properties of the cortical bone as closely as possible because large differences in material properties can lead to implant instability and failure.

TABLE I

| Material | E (GPa) | (MPa) | (%) | K_{1C} (MN m^{-3/2}) |
|---|---|---|---|---|
| Cortical Bone | 7-30 | 50-150 | 1-3 | 2-12 |
| Cancellous Bone | 0.05-.5 | 10-20 | 5-7 | |
| Co—Cr Alloys | 230 | 900-1540 | 10-30 | ˜100 |
| Austenitic Stainless | 200 | 540-1000 | 6-70 | ˜100 |
| Steel | | | | |
| Ti—6Al—4V Alloy | 106 | 900 | 12.5 | ˜80 |
| Alumina | 400 | 450 | ˜.05 | ˜3 |
| Hydroxyapatite | 30-100 | 60-190 | | ˜1 |
| Polyethylene | 1 | 30 | >300 | |

Certain metals and polymers have acceptable biocompatibility as bone implants. Table I compares material properties of various bone replacement materials and cortical bone. However, these and other materials are different from natural bones in various aspects. For example, various metals are much stiffer and stronger than a natural bone and various polymers such as polyethylene are much softer than a natural bone. These and other differences may impose limitations on such metals and polymers for bone implants. As a specific example, these and other metal and polymer materials can withstand much greater strain levels before fracture than a typical natural bone which fractures in a more brittle manner. The loading and unloading occurring with normal human movements may cause permanent elongation of a bone replacement made of such a metal or polymer materials. When such permanent elongation is beyond a certain limit, the bone replacement can become unsuitable for the intended use and need to be removed.

### SUMMARY

This application describes, among others, synthetic materials that are closely compatible in material properties with natural bone materials. The disclosed synthetic materials may be used for a wide range of biomedical applications such as construction of bone replacement parts, dental implants, biocompatible prosthetic interfaces and other structures and devices implanted in a person or animal. Such synthetic materials can also be used as scaffolds to grow cells in vitro for subsequent surgical transfer to a patient.

Examples are described to convert bulk calcium carbonate materials with dense structures of either or both of calcite and aragonite structures into synthetic hydroxyapatite (HAP) materials via a hydrothermal conversion process. For example, bulk pieces of sea-shells or other materials can be directly converted into bulk pieces of dense synthetic materials with hydroxyapatite (HAP) as the main ingredient. The converted bulk pieces are then shaped to make bone implants and other implanted structures and devices. The conversion process uses various raw materials with sufficient or ample supply to produce the desired hydroxyapatite bulk materials. Examples of suitable raw materials include sea-shells, sea urchin spines, and other skeletal materials from marine and other animals. Such raw materials are relatively inexpensive. The conversion process is relatively simple and the converted hydroxyapatite materials are dense bulk pieces and can be directly shaped and processed to construct desired implants without additional processing to alter the underlying material structure. Such dense bulk pieces of hydroxyapatite materials can be manufactured at a reasonable cost due to the low cost of the initial raw materials and the simple conversion process.

In one implementation, an implant fabrication method is described to include using a bulk calcium carbonate material in a dense structure to contact ammonium phosphate in a water solution; and heating the bulk calcium carbonate material and the ammonium phosphate in the water solution to a temperature from about 150° C. to about 250° C. to produce a bulk dense hydroxyapatite material. The bulk calcium carbonate material may be a sea-shell such as a conch shell, a clam-shell, or a abalone shell.

In another implementation, an implant fabrication method us described to include using a piece of bulk marine bone to contact ammonium phosphate in a water solution; and heating the bulk marine bone and the ammonium phosphate in the water solution to a temperature from about 150° C. to about 250° C. to produce a bulk hydroxyapatite material. The piece of marine bone may be a piece of cuttlebone.

In another implementation, an implant material is described to include a bulk hydroxyapatite material, which is produced by a process of contacting a bulk calcium carbonate material in a dense structure of a natural material with ammonium phosphate in a water solution, and heating the bulk calcium carbonate material and the ammonium phosphate in the water solution to a temperature from about 150° C. to about 250° C. to produce the bulk hydroxyapatite material. The bulk calcium carbonate may be a bulk sea-shell piece or a bulk piece of marine bone.

In another implementation, an implant fabrication method is described to include contacting a bulk piece of a sea urchin spine with ammonium phosphate in a water solution; and heating the sea urchin spine and the ammonium phosphate in the water solution to a temperature from about 150° C. to about 250° C. to produce a bulk beta-tricalcium phosphate material.

In another implementation, an implant material is described to include a bulk beta-tricalcium phosphate material which is produced by a process of: contacting a bulk piece of a sea urchin spine with ammonium phosphate in a water solution, and heating the sea urchin spine and the ammonium phosphate in the water solution to a temperature from about 150° C. to about 250° C. to produce the bulk beta-tricalcium phosphate material.

In another implementation, an implant is described to include a bulk beta-tricalcium phosphate material produced from a bulk natural marine material by a hydrothermal conversion process. The conversion may include contacting the bulk natural marine material with ammonium phosphate in a water solution, and heating the bulk natural marine material and the ammonium phosphate in the water solution to a temperature from about 150° C. to about 250° C. to produce the bulk beta-tricalcium phosphate material. The bulk natural marine material may include a sea urchin spine.

In yet another implementation, an implant is described to include a dense hydroxyapatite material converted from a bulk natural marine material by a hydrothermal conversion process. The conversion may include contacting the bulk natural marine material with ammonium phosphate in a water solution, and heating the bulk natural marine material and the ammonium phosphate in the water solution to a temperature from about 150° C. to about 250° C. to produce the dense hydroxyapatite material. The bulk natural marine material may be a bulk piece of a sea-shell.

In yet another implementation, a method for transferring cells to a patient is disclosed. This method includes planting cells harvested from a patient onto a bulk dense hydroxyapatite material substrate to grow and replicate the cells in vitro. The bulk dense hydroxyapatite material substrate is synthesized from a bulk calcium carbonate material in a dense structure by contacting ammonium phosphate in a water solution in a hydrothermal process. This method also includes removing the cells from the bulk dense hydroxyapatite material substrate; and surgically planting the removed cells into the patient.

These and other implementations and examples are described in greater detail in the attached drawings, the detailed description and the claims.

### BRIEF DESCRIPTION OF THE DRAWINGS

FIG. 1 shows the main chemical reaction process and associated the compounds used in the disclosed examples for converting sea-shells and other calcite and aragonite based material with dense structures.

FIGS. 2 through 10 show images and measured data of conch and clam-shells and converted materials after hydrothermal conversions.

### DETAILED DESCRIPTION

Hydroxyapatite (HAP) is a biocompatible material and constitutes the main component of human bones. Hydroxyapatite has desired osteoconductive properties in addition to its biocompatibility and thus can be used in many biological and biomedical applications, including a wide range of implants for human and animals, such as bone graft substitutes, sustained-release drug delivery devices, in-situ purification of proteins and others. Many of these medical implant applications require highly densified implants with a mechanical strength sufficient to substantially retain its shape under force or to bear load.

However, various techniques for converting calcite-based materials into hydroxyapatite produce hydroxyapatite materials in various forms that lack material structures with sufficient rigidity and material strength for above and other applications. Hydroxyapatite powders or hydroxyapatite particulates, for example, are produced by various conversion techniques. As such, additional processing such as molding is further conducted on the hydroxyapatite powder or particulate material to form usable hydroxyapatite bulk pieces.

For example, hydroxyapatite powder was formed by chemical reactions in aqueous solutions by using either calcium deficient hydroxyapatite:
6CaHPO₄+3Ca₄(PO₄)₂O→2Ca_{g}HPO₄(PO₄)₅OH+H₂O
Or 3.66 weight percent carbonated hydroxyapatite: 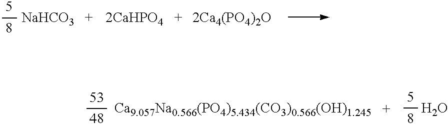
where the reactants were mixed in water and subjected to a pressure of 70 MPa for about 30 seconds to produce hydroxyapatite powder. The hydroxyapatite powder is then molded into desired geometries. After molding, the samples were stored at body temperature (37.4° C.). See, Martin R I, Brown P W. “Mechanical properties of apatite formed by acid-base reactions at 37.4 degrees C.” in IEEE Proceedings of the 1996 Fifteenth Southern Biomedical Engineering Conference (Cat. No.96TH8154), pp. 11-13 (New York, 1996). The liquid to solid ratio was used to determine the porosity of the molded hydroxyapatite samples and the porosity of the molded hydroxyapatite samples approached 80 percent as the liquid to solid ratio approached one to one. Measurements based on mechanical testing for compression and three point bending indicated a logarithmically increasing compressive strength as the porosity of the samples decreases.

As another example, bulk hydroxyapatite samples have also been formed by sintering hydroxyapatite powders at a temperature between 500° C. and 1400° C. See, e.g., Krajewski A, Ravagliolo A, Celotti G, Piancastelli A. “Characterization and annealing of wet prepared synthetic hydroxyapatite powders for high-purity bioceramics”. Crystal Research & Technology, vol.30, no.6, pp.843-52 (Germany, 1995). In another sintering method, calcium hydroxide was mixed with phosphoric acid in a water ammonia solution with a starting pH of over 10.5. The products were filtered, dried, and ground into a powder. This powder was then sintered at 1200° C. See, McKeehan, J. in “Molecular Origins of Bioactivity in Hydroxyapatite Bone Implant Materials”, Doctoral Thesis Proposal, Department of Materials Science and Engineering, Massachusetts Institute of Technology, pp 8-9 (May 12, 2003).

As a further example, Ye Xu el al. reported a technique for hydrothermal conversion of pieces of the marine coral Porites into porous coralline hydroxyapatite (CHA) in “Hydrothermal conversion of coral into hydroxyapatite” in Material Characterization, Vol. 47, pages 83-87 (2001). The coral materials were selected for conversion into hydroxyapatite because the cancellous pore structures in corals are essential for the in-growth of bone tissue and for circulation of body fluids.

In the above and other approaches to conversion of other materials into hydroxyapatite materials in form of powders, particulate materials and porous solids, the initial raw materials lack desired rigid and strong structures similar to bones in human and animals. Marine coral Porites have structurally random channels to form stochastic foam-like structures, are much more porous and brittle and have much rougher texture than natural bones in human and animals. Therefore, the converted porous coralline hydroxyapatite (CHA) from marine coral Porites lacks the mechanical and structural compatibility with natural bones in various aspects such as the material density, rigidity, hardness, strength and smooth surface interface at bone joints. As such, the converted porous coralline hydroxyapatite (CHA), although exhibiting better biocompatibility than many metals and polymers, are not suitable for various bone implants such as implants that bear loads and implants at bone joints. The molding and other techniques for changing powder or particulate hydroxyapatite materials into bulk materials are complicated, expensive and the final bulk material often lack the desired compatibility in structural and mechanical properties with natural bones.

One technical hurdle to synthesis of suitable synthetic bone materials from calcite-based and aragonite-based materials is the difficulty of making structurally dense or structurally rigid forms of hydroxyapatite, structured in a manner that imparts desired mechanical properties. Careful selection of the raw materials is important to producing hydroxyapatite with desired structures and material properties and can simplify the fabrication process. The conversion techniques described in this application avoid using non-bulk materials or porous and brittle materials like marine corals as the raw materials for the conversion and select certain calcite-based and aragonite-based bulk materials with rigid and strong structures. This selected use of raw materials in bulk pieces with desired initial material structures allows for direct conversion from the raw materials into well-structured bulk hydroxyapatite materials without the need for further material processing to achieve desired structural and mechanical properties for implants. Examples of selected calcite-based and aragonite-based raw bulk materials described here include sea-shells with dense and rigid layered structures such as conch, clam and abalone shells, sea urchin spines with layered structures, and cuttlebones with a light weight structure of a high stiffness.

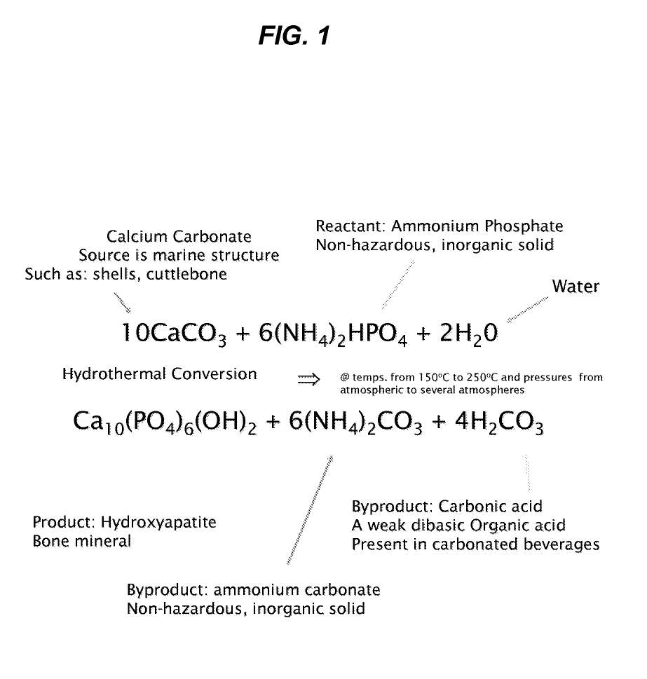

The present techniques for converting bulk calcium carbonate materials into bulk hydroxyapatite materials use a hydro-thermal conversion process. The chemical formula of hydroxyapatite (HAP) can be expressed as:
Ca₍₁₀₋ₓ₎(HPO₄)ₓ(PO₄)₍₆₋ₓ₎(OH)₍₂₋ₓ₎
where the variable x ranges from zero to one. Hydroxyapatite is called calcium deficient hydroxyapatite when x is equal to one (Ca₉HPO₄(PO₄)₅OH), while HAP with x equal to zero is called stoichiometric hydroxyapatite (Ca₁₀(PO₄)₆(OH)₂). The basic conversion process is to contact a bulk calcium carbonate material in a dense or rigid structure with ammonium phosphate in a water solution and then heat the calcium carbonate material and the ammonium phosphate in the water solution to a temperature from about 150° C. to about 250° C. to produce a bulk hydroxyapatite material with a sense or rigid structure. FIG. 1 shows the chemical reaction to produce hydroxyapatite (Ca₁₀(PO₄)₆(OH)₂) from calcium carbonate CaCO₃.

Calcium carbonate in FIG. 1 exists in various forms. The conversion techniques and bulk HAP materials described in this application use bulk calcium carbonate materials with dense or rigid structures to react with ammonium phosphate. The hydro-thermal conversion process can be designed to maintain the architectures of the initial bulk calcium carbonate materials. Many sea-shells (e.g., conch, clams, and abalone) are formed of calcite and aragonite crystals and have dense, tailored hierarchical structures with excellent mechanical properties for bone implants such as bone replacements and implant interfaces. Various marine bones, such as bulk cuttlebones, have regular orthogonal cavity arrays to form a rigid light-weight structure with a high stiffness. Sea urchin spines have concentric cylindrical layers to form light-weight rigid structures with an averaged density of about 1.25 gm/cm³. These natural structures inherently have desired mechanical and structural properties for various implants. For example, dense sea-shells can be converted into dense HAP materials that closely match the density, structure and mechanical properties of natural bones. Bulk HAP materials converted from bulk cuttlebones and sea urchin spines may be used to construct rigid and light-weight implants. The superior properties of the converted HAP from sea-shells, marine bones, and sea urchin spines are derived from the natural architectures of the layered structures in these materials. For example, abalone shells have hexagonal calcite tiles separated by a protein layer that imparts significant damage tolerance to these shells. The soft nature of the cuttlebones allows the converted HAP from cuttlebones to specific shapes for implants. The calcite to hydroxyapatite conversion can be carried out after the cuttlebones are shaped because the converted HAP material is significantly harder that the natural cuttlebones. Similarly, bulk sea-shell pieces may be machined into desired shapes prior to the hydro-thermal conversion process. Sea-shells, sea urchin spines and cuttlebones are available in large quantities and relatively inexpensive. For example, cuttlebones are regularly sold for $5 per pound.

The conversion shown in FIG. 1 can be conducted in a simple reaction vessel at temperatures up to 250° C. at a relatively low cost. The processing temperature is sufficiently high to destroy any known bacteria that might be on the starting material and thus converted HAP materials are biocompatible. The natural structures of the initial bulk materials such as sea-shells are preserved by the conversion process and the structures of the converted HAP bulk pieces are ready for use and no additional material processing for modify the structures is needed. For example, bulk HAP materials converted from sea-shells are fully dense and there is no need for any secondary densification operation to increase the density of the converted HAP materials.

The natural structure of the sea urchin spines, although made of aragonite and calcite crystals like sea-shells and cuttlebones, is slightly different from the structure of aragonite and calcite crystals found in sea-shells and cuttlebones and some other natural materials. Hence, instead of being converted into hydroxyapatite shown in FIG. 1, sea urchin spines, in reaction with ammonium phosphate solution under a heated condition, are transformed into a different synthetic bone material: beta-tricalcium phosphate (β-TCP). The beta-TCP is closely related to the hydroxyapatitie structure and is a natural material comprised of calcium and phosphorous which are the primary constituents of bones. For example, a beta-TCP material may include 39% calcium and 20% phosphorous by weight in a molar ration of 1.5: Ca₃(PO₄)₂. Notably, beta-TCP is both bio-compatible and bio-absorbable and hence can be used to manufacture a compatible implant that eventually is absorbed into the body over time. Dense beta-TCP materials can be used for a variety of implants in animals and humans.

The following sections describe specific examples for conversion of bulk pieces of conch and claim shells with naturally dense and rigid layer structures into dense bulk HAP materials. Strombus gigas (conch) shells and Tridacna gigas (Giant clam) shells have dense, tailored structures that impart excellent mechanical properties to these shells. Conch and clam seashells were converted to bulk pieces of dense hydroxyapatite (HAP) by a hydrothermal method at different temperatures and for different conversion durations. High temperatures were found to accelerate the conversion process, however, cracks were found on the surface of the samples converted at high temperatures or for very long conversion times. The conversion at 180° C., refreshing the diammonium hydrogen phosphate [(NH₄)₂HPO₄] solution every 2 days, produced samples of good quality. Different morphologies of HAP were found in different regions of the converted shells, which may be caused by different structural morphologies and in different amounts of porosity in the original shells. Partially converted shell samples with dense HAP layers on the surface growing inward and original shell structures inside have an average fracture stress about 137-218 MPa, which is close to the mechanical strength of compact human bone.

The starting materials in our experiments were diammonium hydrogen phosphate [(NH₄)₂HPO₄], S. gigas (conch) shells and T. gigas (Giant clam) shells. Conch shells and claim shells have dense layered structures with averaged densities of 2.81 and 2.84 gm/cm³, respectively. These shells were cut into small bulk pieces, about 28 mm×20 mm×3 mm, cleaned with deionized water and put in an autoclave. The autoclave was filled with a specified concentration of a (NH₄)₂HPO₄ solution. The autoclave was sealed and heated at different temperatures for different durations. The starting concentration of (NH₄)₂HPO₄ solution ranged from 0.12 g/ ml to 0.6 g/ml, and temperatures from 180° C. to 240° C. The best conversion results in the conducted conversions were achieved for the starting (NH₄)₂HPO₄ solution at 0.2 g/ ml solution and at a temperature of 180° C., where the (NH₄)₂HPO₄ solution was refreshed within the autoclave every 2 days.

Sample phases were identified using X-ray diffraction (XRD), while the morphology of the structures was studied by optical microscopy, scanning electron microscopy (SEM) and transmission electron microscopy (TEM). The element compositions of the converted phases were identified by qualitative energy dispersive X-ray spectroscopy (EDS) in both the SEM and TEM. Quasi-static compression tests with samples about 5 mm×4 mm×3 mm were conducted on a servohydraulic test frame at a strain rate of 2×10⁻³/s.

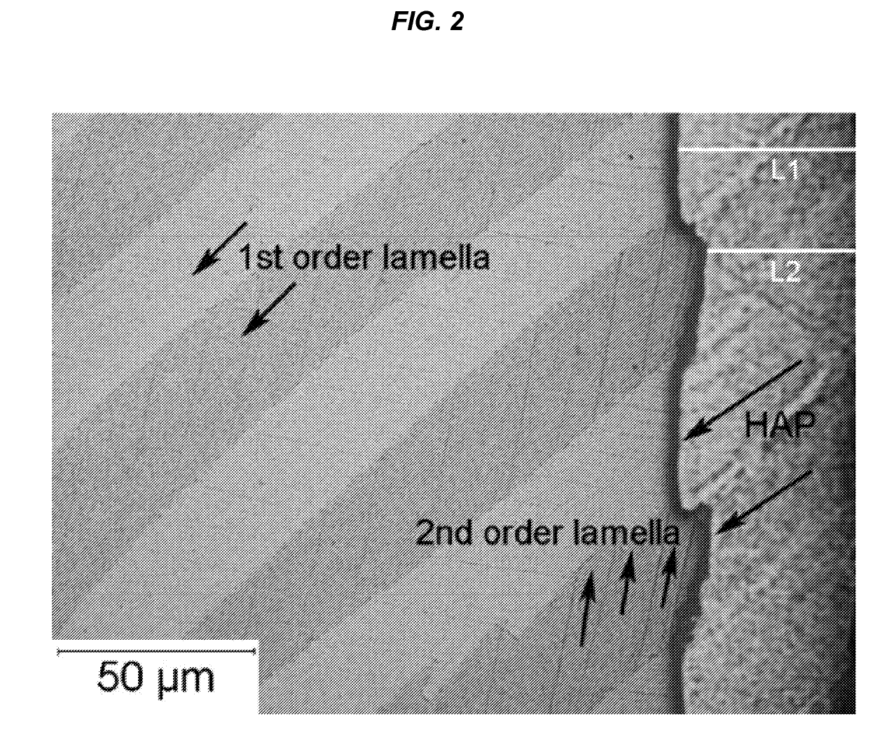

The original conch shell had three layering scales, composed of first-order lamellae, which are subdivided into second-order lamellae, and further subdivided into third-order lamellae. FIG. 2 shows the optical photograph of polished section of conch converted at 180° C. for 1 day. The first-order lamellae was measured to be about 30 μm thick and second-order lamellae was measured to be about 10-20 μm wide. The HAP layers grew inward replacing first-order lamellae of the conch shell.

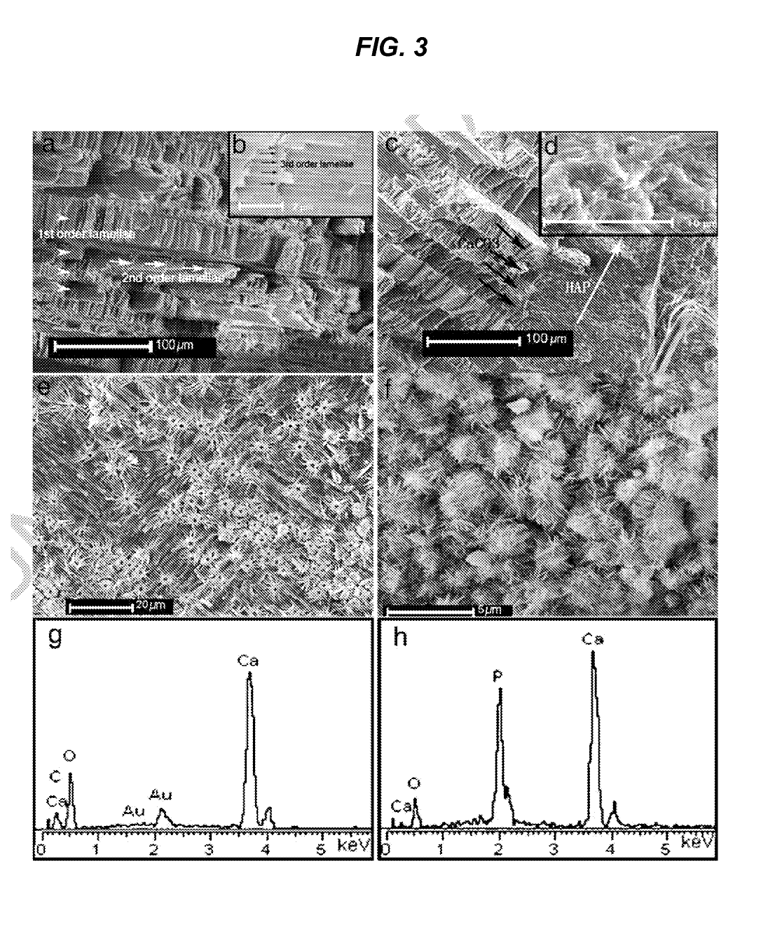

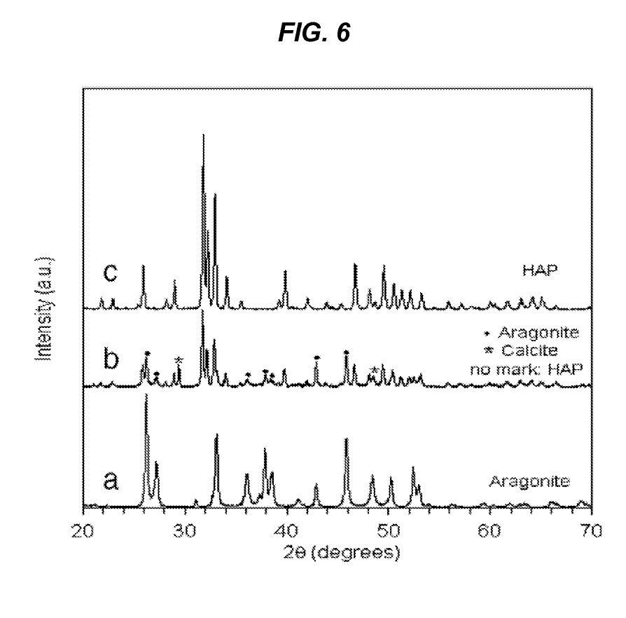

FIG. 3 shows the scanning electron microscopy (SEM) images of the original and converted conch shells and related energy dispersive spectrometry (EDS) spectra. FIG. 3a shows the structures of the macro-layer of the conch shell. The first-order lamellae, composed of many second-order lamellae formed by third-order lamellae, are visible in FIGS. 3a and 3b. FIG. 3c is the SEM image of conch converted at 180° C. for 10 days. The left portion of the image is from the original shell (marked with black arrows). The HAP layer (marked with white arrow), which has a very dense structure (FIG. 3d), has replaced the CaCO₃ through the hydrothermal reaction. The components of these two phases were identified by EDS spectra in FIGS. 3g and 3h. The EDS spectrum from the HAP portion (FIG. 6h) shows the characteristic peak of phosphorus. FIGS. 3e and df are images from HAP portions of conch converted at 200° C. for 10 days. Dense structures with some small pores are shown in FIG. 3e. These pores are present in the original conch, altering the HAP morphology created by the conversion. FIG. 3f reveals some HAP crystals assembled together to form spheres. Comparing FIGS. 3d, 3e, and 3f, different morphologies of HAP were found in different parts of samples after hydrothermal conversion. The different structural morphologies are likely the result of different solid-gas, solid-liquid, and solid-solid interface conditions during conversion of aragonite crystals to HAP.

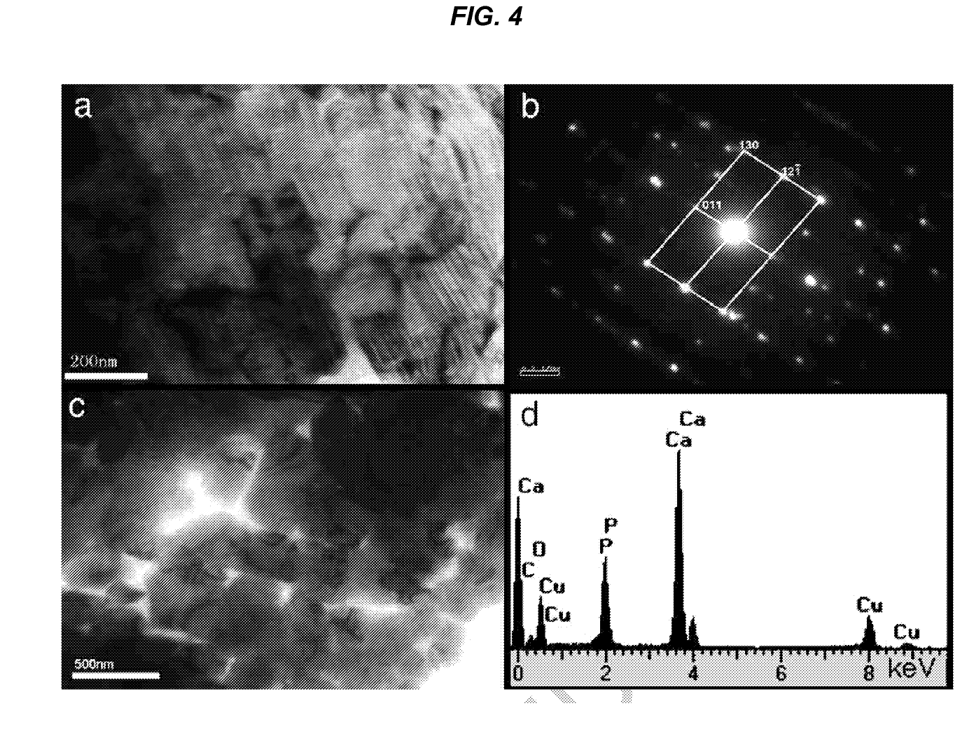

FIG. 4 shows the TEM images and electron diffraction of the original conch shell in FIGS. 4a and 4b. FIG. 4a is a TEM image of aligned third-order lamellae in the conch shell with a scale of about 20-30 nm thicknesses in each lamellae. In the electron diffraction pattern of this sample (FIG. 4b), most diffraction spots can be associated with the aragonite crystals, and their twin lamella aligned along the [ 3̅1 1̅] zone axis. The crystal planes from aragonite were marked in the pattern. After conversion at 180° C. for 4 days, particles about 300 nm were observed as shown in FIG. 4c. The characteristic peak of phosphorus, along with peaks for calcium, carbon and oxygen indicative of the HAP phase, is shown in the EDS spectrum presented in FIG. 4d, taken from the region shown in FIG. 4c.

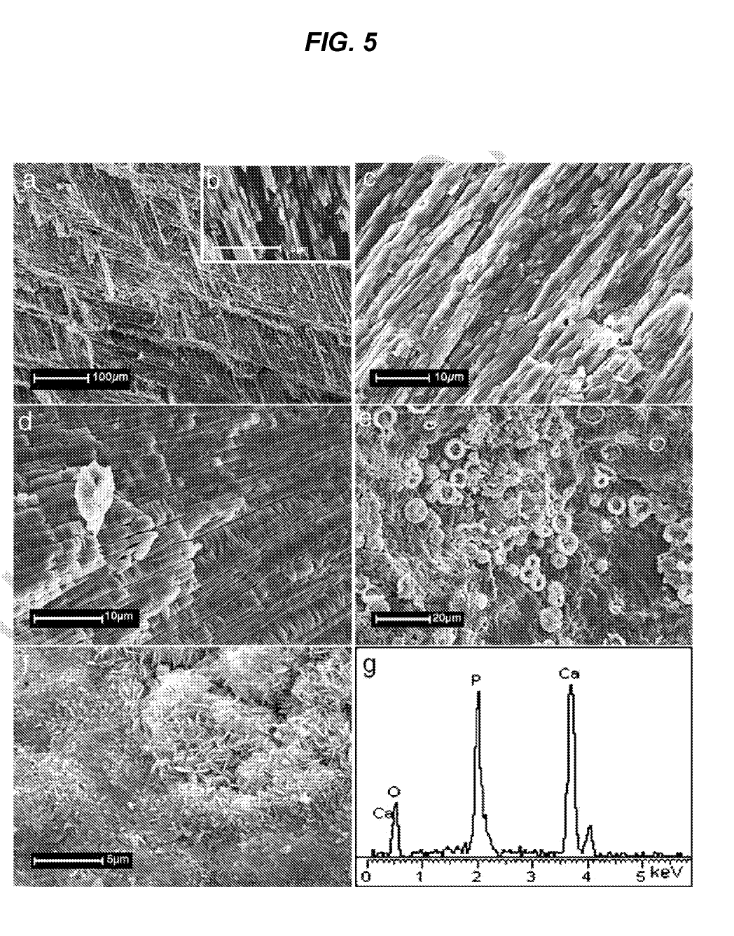

FIG. 5 shows SEM images of the original clam-shell, the converted clam, and related EDS spectrum. The original clam-shell has many layers (FIG. 5a), which are composed of small, aligned CaCO3 plates (FIG. 5b), while other parts of the original clam-shell are composed of larger, thin CaCO₃ plates (FIG. 5c). FIG. 5d is an image from clam converted at 180° C. for 20 days, which has the similar morphology to FIG. 5c. The EDS spectrum (FIG. 5g) from layers in FIG. 5d indicates that they are composed of HAP. This result indicates that the bulk of the aragonite to HAP conversion has occurred preserving the original structural morphology of the shell (i.e. layered aragonite to layered HAP). FIG. 5e is the HAP from portion of the same sample as FIG. 5d and f is the HAP in the clam converted at 180° C. for 12 days, wherein some porosity was found. It was observed that when the initial shell structure is very dense, the converted HAP shell is also dense. However, when pores are present, the converted materials have a range of less dense morphologies.

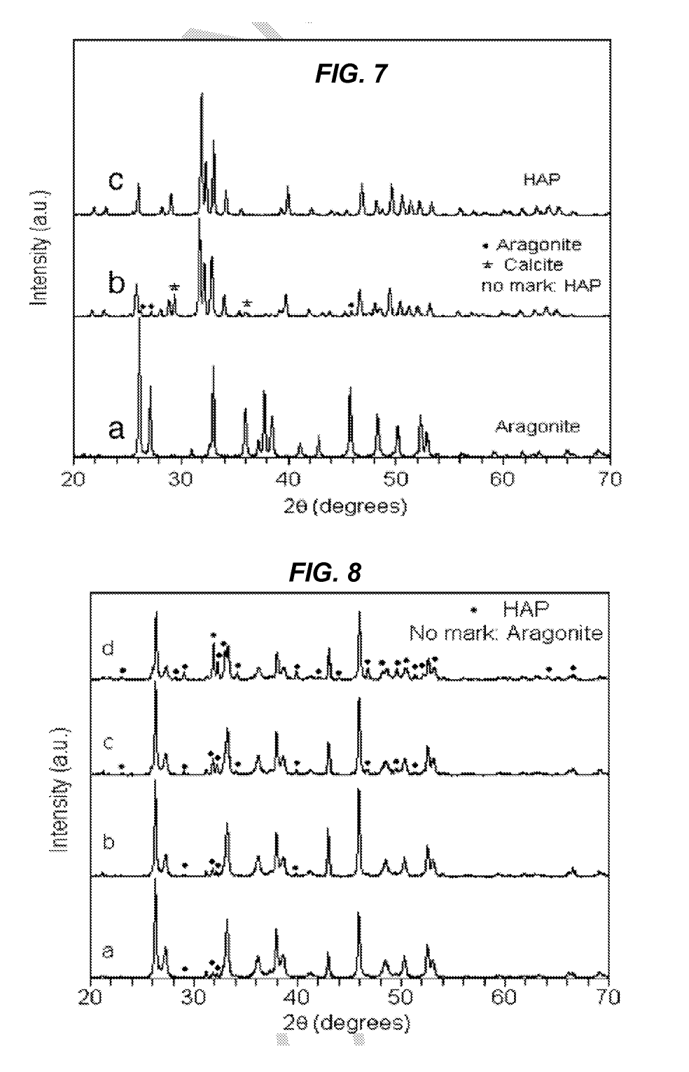

FIG. 6 shows bulk XRD patterns of original conch shell and the conch shell hydrothermally converted at 180° C. and 200° C. for 10 days. FIG. 7 shows bulk XRD patterns of the original clam-shell and the clam shell hydrothermally converted at 180° C. and 200° C. for 10 days in the autoclave. The original conch and clam-shells are mainly composed of aragonite crystals (FIGS. 6a and 7a) with organic matrices. Small amount of calcite crystals was found in some shell samples. After undergoing the conversion at 180° C. for 10 days, the conch and clam-shells were almost entirely converted to HAP. A small amount of unconverted aragonite crystals remained, and some aragonite crystals transformed into calcite (FIGS. 6b and 7b). The conch and clam-shells were completely converted to HAP at 200° C. for 10 days (FIGS. 6c and 7c). Comparison of FIG. 6b with FIG. 6c and comparison of FIG. 7b with FIG. 7c indicate that the conversion process was accelerated at higher temperatures. In these experiments, 110 small cracks were formed on the surfaces of shells converted at temperatures above 200° C.

Additional conversion tests were conducted. For example, a batch of 7 pieces of conch shells and 7 pieces of clam shells were mixed together in a 250 ml solution of 0.16 g/ml (NH₄)₂HPO₄ in an autoclave. The autoclaved was then sealed and heated at 180° C. The (NH₄)₂HPO₄ solution was refreshed every 2 days. Pieces of conch and clam were collected from the autoclave after 2, 4, 6, 8, 12, 16 and 20 days. FIG. 8 shows XRD results of conch converted at different times: 2 days (FIG. 8a), 4 days (FIG. 8b), 8 days (FIG. 8c) and 20 days (FIG. 8d). After conversion for 2 days (FIG. 8a), a small amount of aragonite crystals were converted into HAP. With increasing conversion time, more HAP peaks appear, with greater intensities in the diffraction patterns (FIGS. 8b, 8c and 8d). This indicates that increasing amounts of aragonite crystals were converted to HAP. The similar result was found for conversion of clams in this experiment. A few cracks were found on the surfaces of samples converted for 16 days and 20 days.

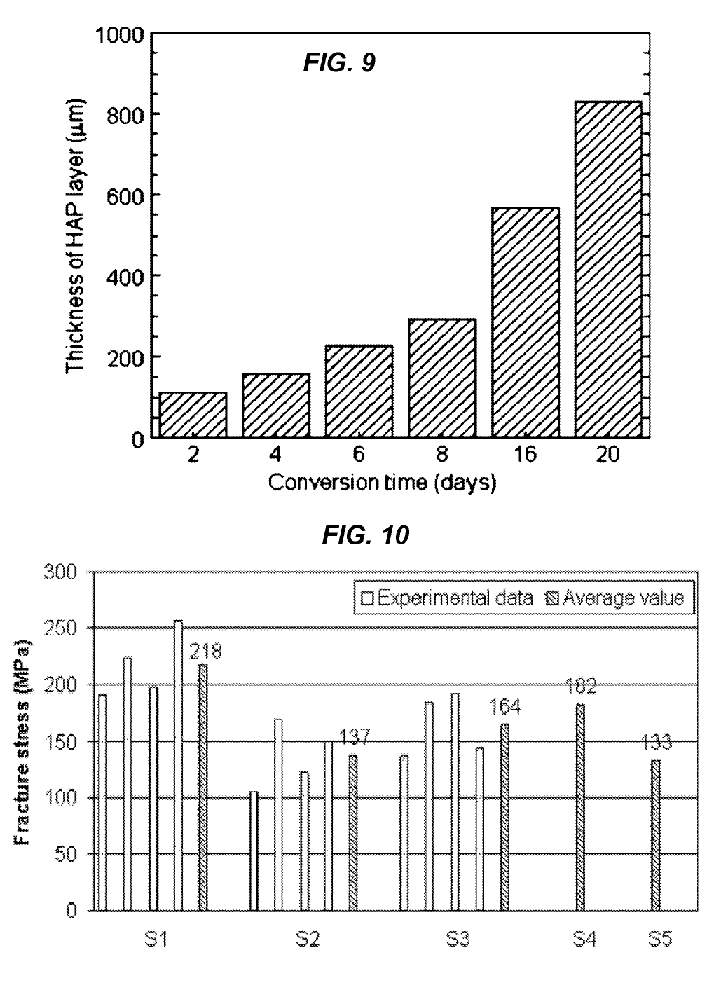

In order to measure the thickness of the converted HAP layers, bulk pieces of about 3 mm thick from the middle portion of each converted shell were sectioned. These pieces were then mounted and polished. Polished pieces were observed and measured under an optical microscope. The total thickness of HAP layers on the sample surface was measured directly from the images, and the thickness of the HAP layers within the converted conch samples at different conversion times at a temperature of 180° C. are shown in FIG. 9. The measurement of thickness of the HAP layer on one surface of the shell is schematically shown in FIG. 2. Fifteen lines (such as 137 lines: L1, L2 in FIG. 2) were drawn across the thickness of the HAP layer, and the average length of the 15 lines was calculated. The average thickness of HAP layer on the opposite surface was calculated by the same method. The summed thickness of these two HAP layers on these surfaces was then used as the total thickness of the HAP layers on converted conch shell in FIG. 9.

The thickness data in FIG. 9 suggests that, the amount of CaCO₃ crystals that are converted into HAP increases with the reaction time, more CaCO₃ crystals were converted to HAP, in agreement with the XRD results. After 20 days, HAP layers about 830 μm thick were formed on the converted conch sample.

In addition, quasi-static compression tests were performed in the converted conch shells by applying the compression normal to the HAP/CaCO₃ layer at a strain rate of 2×10⁻³/s. FIG. 10 shows the fracture stresses of three different conch samples (about 5 mm×4 mm×3 mm) converted at 180° C., and that of compact human bone. The data represented by S1, S2 and S3 was measured for conch shells converted at 180° C. for 4 says, 8 days and 20 days, respectively. The data represented by S4 and S5 was measured averaged compression strength of compact human bone located in two different directions: apparel to and normal to the bone axis, respectively. The average fracture stresses for the three conch samples were 218, 137 and 164 MPa, respectively. The compression strength of the compact human bone is about 170-193 MPa when loaded parallel to the bone axis, and about 133 MPa when loaded normal to the bone axis. Shell samples, after being partially converted to hydroxyapatite (HAP), have mechanical strength values very similar to the compact human bone.

The synthetic materials converted from sea shells and other natural materials described in this application may be used as a substrate for growing various cells such as chondrocytes and the grown cells are then harvested and transferred into the body of a patient. In this application, the synthetic material is not implanted into the patient. The porous surface of the hydroxyapatite material converted from, e.g., a bulk sea urchin spine material, is used to attach and grow the cells, such as cartilage cells or other connective tissue cells. In this application, the hydroxyapatite material substrate is a caffold for delivery and retention of chondrogenic cells in cartilage tissue engineering. Beyond serving as a mechanical substrate, the scaffold can interact with cells, bioactive molecules, and mechanical signals in a dynamic and synergistic manner to direct and organize the process of regeneration. The cells can be either embedded in microporous scaffolds such as hydrogels or seeded into the pores of three-dimensional macroporous scaffolds. The scaffold should be sterilizable, biocompatible, biodegradable, possess sufficient mechanical strength, and support cell differentiation and cartilage matrix production. The size and shape of the tissue to be regenerated, the nature and type of cartilage defect, and the conditions of the host should also be considered when selecting a scaffold.

As an example, the entire surgical transfer process can include a cell growth stage and a cell transfer stage. In the cell growth stage, chondrocytes of the patient are removed arthroscopically from a non load-bearing area (e.g., either the intercondylar notch or the superior ridge of the medial or lateral femoral chondyles). These removed cells are placed on a synthetically-made hydroxyapatite material substrate or scaffold and are grown in vitro for a a period of time to replicate and grow into a sufficient number of cells. Initial 10,000 cells tha that are originally harvested can reach the population of 10-12 million cells in approximately six weeks. The scaffold material can enhance the growth rate of the cells, thus reducing the period between extracting the cells and replacing the proliferated cells. After this cell proliferation period, the patient undergoes a second surgery in which the millions of chondrocytes are surgically injected into the patient. These injected cells can be held in place on the cartilage defect by a periosteal flap, for example, and a small piece of bone tissue can be sutured over the damaged area. The implanted chondrocytes can then divide and integrate with surrounding tissue under the flap and potentially generate hyaline-like cartilage.

While this specification contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

Only a few examples are described. Other variations and enhancements may be made based on what is described here.

What is claimed is

1. An implant fabrication method, comprising:
using a bulk calcium carbonate material in a dense structure to contact ammonium phosphate in a water solution; and
heating the bulk calcium carbonate material and the ammonium phosphate in the water solution to a temperature from about 150° C. to about 250° C. to produce a bulk dense hydroxyapatite material.

2. The method as in claim 1, further comprising applying a pressure greater than the atmosphere pressure to the bulk calcium carbonate material and the ammonium phosphate in the water solution.

3. The method as in claim 1, wherein the bulk calcium carbonate material is a sea shell.

4. The method as in claim 3, wherein the sea shell is a conch shell, a clam shell, or a abalone shell.

5. An implant fabrication method, comprising:
using a piece of bulk marine bone to contact ammonium phosphate in a water solution; and
heating the bulk marine bone and the ammonium phosphate in the water solution to a temperature from about 150° C. to about 250° C. to produce a bulk hydroxyapatite material.

6. The method as in claim 5, wherein the piece of marine bone is a piece of cuttlebone.

7. An implant material, comprising a bulk hydroxyapatite material, which is produced by a process of:
contacting a bulk calcium carbonate material in a dense structure of a natural material with ammonium phosphate in a water solution, and
heating the bulk calcium carbonate material and the ammonium phosphate in the water solution to a temperature from about 150° C. to about 250° C. to produce the bulk hydroxyapatite material,
wherein the bulk calcium carbonate is a bulk sea shell piece or a bulk piece of marine bone.

8. An implant fabrication method, comprising:
contacting a bulk piece of a sea urchin spine with ammonium phosphate in a water solution; and
heating the sea urchin spine and the ammonium phosphate in the water solution to a temperature from about 150° C. to about 250° C. to produce a bulk beta-tricalcium phosphate material.

9. The method as in claim 8, further comprising applying a pressure greater than the atmosphere pressure to the sea urchin spine and the ammonium phosphate in the water solution.

10. An implant material, comprising a bulk beta-tricalcium phosphate material, which is produced by a process of:
contacting a bulk piece of a sea urchin spine with ammonium phosphate in a water solution, and
heating the sea urchin spine and the ammonium phosphate in the water solution to a temperature from about 150° C. to about 250° C. to produce the bulk beta-tricalcium phosphate material.

11. The material as in claim 10, wherein the process further comprises:
applying a pressure greater than the atmosphere pressure to the sea urchin spine and the ammonium phosphate in the water solution.

12. An implant, comprising a bulk beta-tricalcium phosphate material produced from a bulk natural marine material by a hydrothermal conversion process.

13. The implant as in claim 12, wherein the conversion comprises:
contacting the bulk natural marine material with ammonium phosphate in a water solution, and
heating the bulk natural marine material and the ammonium phosphate in the water solution to a temperature from about 150° C. to about 250° C. to produce the bulk beta-tricalcium phosphate material.

14. The implant as in claim 13, wherein the bulk natural marine material includes a sea urchin spine.

15. An implant, comprising a dense hydroxyapatite material converted from a bulk natural marine material by a hydrothermal conversion process.

16. The implant as in claim 15, wherein the conversion comprises:
contacting the bulk natural marine material with ammonium phosphate in a water solution, and
heating the bulk natural marine material and the ammonium phosphate in the water solution to a temperature from about 150° C. to about 250° C. to produce the dense hydroxyapatite material.

17. The implant as in claim 16, wherein the bulk natural marine material includes a bulk piece of a sea shell.

18. A method for transferring cells to a patient, comprising:
planting cells harvested from a patient onto a bulk dense hydroxyapatite material substrate to grow and replicate the cells in vitro, wherein the bulk dense hydroxyapatite material substrate is synthesized from a bulk calcium carbonate material in a dense structure by contacting ammonium phosphate in a water solution in a hydrothermal process;
removing the cells from the bulk dense hydroxyapatite material substrate; and
surgically planting the removed cells into the patient.

19. The method as in claim 18, wherein:
the cells are chondrocyte cells.
